(19) United States
(12) Patent Application Publication
(10) Pub. No.: US 2022/0196684 A1
(43) Pub. Date: Jun. 23, 2022

(54) METHOD FOR DETECTING OR MONITORING THE DEVELOPMENT OF A CHRONIC DEGENERATIVE DISEASE BY IMMUNOASSAY

(71) Applicant: POLYNEUROS, SAINT JEAN D'ILLAC (FR)

(72) Inventors: Michel GEFFARD, Talence (FR); Jean-Pascal ZAMBAUX, Bouliac (FR)

(21) Appl. No.: 17/600,738

(22) PCT Filed: Apr. 1, 2020
(86) PCT No.: PCT/EP2020/059218

§ 371 Date: Oct. 1, 2021

(30) Foreign Application Priority Data
Apr. 1, 2019 (FR) FR1903451

(51) Int. Cl. G01N 33/68; G01N 33/569

(52) U.S. Cl. CPC G01N 33/6896; G01N 33/56916; G01N 2333/21; G01N 2800/2821; G01N 2333/26; G01N 2800/2835

(57) ABSTRACT

The invention relates to an ex vivo method for detecting or monitoring the progression of a chronic degenerative disease, in a sample of human or animal biological fluid, by immunoassay for the presence of antibodies in the sample, including at least:
one or more antibodies directed against at least one endobacterium, and
one or more antibodies directed against a tryptophan oxidation product.

The invention also relates to a kit for implementing such a method.

### CROSS-REFERENCE TO RELATED APPLICATIONS

This application is a U.S. National Stage Application of PCT/EP2020/059218 assigned the international filing date of Apr. 1, 2020 and claiming the benefit of priority from EP patent application FR1903451 filed Apr. 1, 2019, the disclosure of these applications is herein incorporated by reference.

### TECHNICAL FIELD

The invention relates to the ex vivo detection and monitoring of the progression of chronic degenerative diseases in humans or animals.

### BACKGROUND

Degenerative diseases are non-infectious and non-contagious pathologies, characterized by the progressive disruption of the normal functions of the body, and especially of the immune system. These are therefore progressive multifactorial disorders that lead to very debilitating impairments and disabilities in those affected by them. The causes identified are varied—presence of an unacceptable or toxic substance, accumulation, loss or alteration of a biological substance, hereditary causes, etc.—and have the effect of gradually degrading the cells, tissues or organs concerned. These may be neurodegenerative diseases such as Alzheimer's disease, Parkinson's disease or amyotrophic lateral sclerosis, or degenerative diseases affecting organs or tissues such as heart failure, emphysema, lupus, muscular dystrophy, or eye and nerve degeneration.

In addition to having the characteristic of being degenerative, proliferative and/or autoimmune processes can develop during the progression of the disease. This is called comorbidity, which corresponds to the result of the association between degenerative disease and autoimmune and/or proliferative disease.

The therapeutic solutions provided for degenerative diseases are products that act only on the symptoms, but not on the etiological factors of the disease. It may for example be Riluzole® or Radicava® for Amyotrophic Lateral Sclerosis, Carbidopa® for Azilect® or Mirapex® for Parkinson's, or Aricept®, Donepezil® or Exelon® for Alzheimer's. These drugs have very limited or no effectiveness, are generally toxic and have significant side effects.

In addition, treatments are often ineffective in the very frequent cases where degenerative diseases are diagnosed too late to be able to stop or slow the progression of the disease in time. Indeed, there is no diagnostic test, such result that it is done by trial and error and by elimination, which can take several months or even years.

In addition, since these pathologies are chronic, they require regular monitoring of the progression of the disease, and there is currently no simple and reliable solution that allows such monitoring.

### SUMMARY

The objective of the invention is to overcome the drawbacks of the prior art by proposing a method for both detecting and monitoring the progression of a chronic degenerative disease in humans or animals, which is simple to implement, minimally invasive, fast, efficient and reliable.

To this end, the object of the invention is an ex vivo method for detecting or monitoring the progression of a chronic degenerative disease, in a sample of human or animal biological fluid, preferably of human or animal plasma and/or serum, by immunoassay for the presence of antibodies in the sample, including at least:
one or more antibodies directed against at least one enterobacterium, that is to say, against at least one component of at least one enterobacterium, and
one or more antibodies directed against a tryptophan oxidation product.

The method can also comprise the immunoassay for the presence in the sample of other antibodies, including:
at least one antibody directed against a product associated with lipoperoxidation mechanisms (also called “lipoperoxidation product”), and/or
at least one antibody directed against a nitrated or nitrosylated product, and/or
at least one antibody directed against a catecholamine oxidation product, and/or
at least one antibody directed against a bacterial metabolism product.

Advantageously, this method is simple to implement, since it is carried out on a sample of biological fluid and it implements conventional immunoassay techniques. The immunoassay method mimics ex vivo what happens in vivo where antigens are bound to immunoglobulins (antibodies).

The method according to the invention can be implemented using a kit that constitutes another object of the invention.

Other features and advantages of the invention will emerge from the detailed description that follows.

### DESCRIPTION OF THE FIGURES

FIG. 1 shows the results of the detection of circulating antibodies directed against Citrobacter koseri in IgA, IgM and IgG, Klebsellia oxytoca in IgA, IgM and IgG and Klebsellia pneumoniae in IgA, IgM and IgG, during the progression of Amyotrophic Lateral Sclerosis, as a distribution of a population of ALS patients; the midpoint represents 50% of the population.

FIG. 2 shows the results of the detection of circulating antibodies directed against azelaic acid in IgA, IgM and IgG, oleic acid in IgA, IgM and IgG and myristic acid in IgA, IgM and IgG, during the progression of Amyotrophic Lateral Sclerosis, as a distribution of a population of ALS patients; the midpoint represents 50% of the population.

FIG. 3 shows the results of the detection of circulating antibodies directed against kynurenic acid in IgA, IgM and IgG, xanthurenic acid in IgA, IgM and IgG and anthranilic acid in IgA, IgM and IgG, during the progression of Amyotrophic Lateral Sclerosis, as a distribution of a population of ALS patients; the midpoint represents 50% of the population.

FIG. 4 shows the results of the detection of circulating antibodies directed against Pseudomonas Tolaazii in IgA and IgM, Pseudomonas Stutzerii in IgA and IgM, Pseudomonas fluorescens in IgA and IgM and Pseudomonas reactans in IgA and IgM, during the progression of Parkinson's disease, as a distribution of a population of Parkinson's disease patients; the midpoint represents 50% of the population.

FIG. 5 shows the results of the detection of circulating antibodies directed against 5-methoxytryptophol in IgA, IgG and IgM, 5-hydroxytryptophol in IgA, IgG and IgM, and anthranilic acid in IgA, IgG and IgM, during the progression of Parkinson's disease, as a distribution of a population of Parkinson's disease patients; the midpoint represents 50% of the population.

FIG. 6 represents the results of the detection of circulating antibodies directed against NO-creatinine in IgA and IgM, NO-tryptophan in IgA and IgM, NO-phenylalanine in IgA and IgM and NO-histidine in IgA and IgM, during the progression of Parkinson's disease, as a distribution of a population of Parkinson's disease patients; the midpoint represents 50% of the population.

FIG. 7 shows the results of the detection of circulating antibodies directed against NO-creatinine in IgA, IgG and IgM, NO-tyrosine in IgA, IgG and IgM, and NO2 tyrosine in IgA, IgG and IgM, during the progression of Alzheimer's disease, as a distribution of a population of Alzheimer's disease patients; the midpoint represents 50% of the population.

FIG. 8 shows the results of the detection of circulating antibodies directed against xanthurenic acid in IgA, IgG and IgM, anthranilic acid in IgA, IgG and IgM, and kynurenic acid in IgA, IgG and IgM, during the progression of Alzheimer's disease, as a distribution of a population of Alzheimer's disease patients; the midpoint represents 50% of the population.

### DEFINITIONS

Within the meaning of the invention, “circulating antibodies” refers to antibodies that are found in human or animal biological fluids, in particular in the blood, serum and/or plasma of humans or animals.

Within the meaning of the invention, “reagent blank” refers to a solution or a mixture containing all the reagents used during the method according to the invention, but which does not contain the sample to be analyzed. It allows a basic correction to be made to the results of the assay. It defines the background noise of the assay method.

Within the meaning of the invention, “biological fluid” refers to fluid from the body of a human being or of an animal, in particular blood, serum and/or plasma, cerebrospinal fluid, pleural fluids and intraperitoneal, intra-articular fluids, saliva or urine.

Within the meaning of the invention, “degenerative disease” refers to a disease in which degeneration of at least one tissue or degeneration of cells has occurred.

Within the meaning of the invention, “neoantigen” refers to the result of a covalent bond between a radical compound (NO, NO₂, etc.) or a compound associated with the mechanisms of lipoperoxidation (Malondialdehyde, Azelaic acid, etc.) or resulting from the hyperproduction of metabolic compounds (derivatives of tryptophan), and an endogenous cellular or tissue component. A neoantigen is also the result of unmasking a cellular component (fatty acid, phosphatidylinositol, etc.) not physiologically accessible to the immune system that, following active inflammatory processes, is exposed to the immune system.

Within the meaning of the invention, “lipoperoxidation product” or “product associated with lipoperoxidation mechanisms” refers to a product that is at the origin of or results from the peroxidation of lipids, i.e. the oxidation of unsaturated lipids, either by radical species of oxygen and nitrogen, or catalyzed by enzymes. The peroxidation of lipids results in their degradation into products called lipoperoxidation products.

Within the meaning of the invention, “nitrated or nitrosylated product” refer to neoantigens resulting from an overproduction of NO and/or peroxynitrite. This hyperproduction results from the activation of inducible NO synthase, mainly in the cells of the innate immune system, by bacteria, viruses, pollutants, etc.

Within the meaning of the invention, the term “tryptophan oxidation product” refers to a metabolite resulting from the degradation of tryptophan, after the enzymatic activation of the Indolamine-2,3-Dioxygenase (IDO)-1 and/or THO (Tryptophan hydroxylase) pathways. This enzymatic activation takes place in mono-macrophagic cells under the action of bacterial, viral, mycotic, pollutant, and/or pro-inflammatory cytokine and chemokine components.

“Bacterial components” refer to a bacteria degradation product that results from bacterial mechanical or biochemical lysis.

“Bacterial metabolism products” refers to molecules produced by the metabolism of bacteria, the metabolism of a bacterium being the set of biochemical reactions put into play by the bacterium in order to grow.

Within the meaning of the invention, “catecholamine oxidation product” refers to oxidized catecholamines linked to endogenous proteins and associated with N-acetylcysteine (NAC).

Within the meaning of the invention, “ALS” refers to amyotrophic lateral sclerosis.

### DETAILED DESCRIPTION OF THE INVENTION

The object of the invention is therefore an ex vivo method for detecting or monitoring the progression of a chronic degenerative disease.

The disease can be any chronic degenerative disease, and it can in particular be a neurodegenerative disease, in particular ALS, Parkinson's disease or Alzheimer's disease.

The method is carried out ex vivo, outside the human or animal body, in a sample of human or animal biological fluid, preferably of human or animal serum or plasma, which was taken before the implementation of the method. The sample of biological fluid is a sample that has been taken and stored according to the usual standards known to those skilled in the art.

The method according to the invention is carried out by immunoassay for the presence in the sample of circulating antibodies directed against specific antigens of chronic degenerative diseases.

The method can be any type of immunoassay method. It may in particular be an ELISA assay, strip tests, tests carried out using magnetic, latex and/or silica beads, or else a Western blot test. These tests can be implemented according to the knowledge of a person skilled in the art.

In the implementation of the method according to the invention:
The antigens and neoantigens used to implement the method are preferably antigens synthesized ex vivo. In the case where the antigens are bacterial components, they can be obtained from bacterial cultures.
If the antigens used are not bacteria, they are preferably coupled (conjugated) to a protein, such as Bovine Serum Albumin for example, to facilitate the attachment of the antigen to the support (plate, strip, etc. depending on the assay method); thus, an antibody directed against a neoantigen within the meaning of the present invention can mean (excluding bacteria) an antibody directed against a conjugated neoantigen, whether or not the expression “conjugated” is indicated after the neoantigen.
The secondary antibodies, called anti-isotypes, are preferentially conjugated to an enzyme such as peroxidase or alkaline phosphatase are diluted according to their isotypy, in a diluting buffer, called preservation buffer, containing stabilizing proteins. These antibodies are preferably known antibodies that can be purchased.

The method according to the invention preferably comprises an enzyme immunoassay based on the ELISA method. The immunoassay method can comprise the following steps:
the antigens against which the antibodies to be detected in the sample are directed are adsorbed (and optionally dried) on microtiter plates; this step consists in sensitizing microtiter plates with the antigen(s) against which the antibodies, the presence of which is to be detected in the sample, are directed.

The plates are then either used directly to implement the method, or dried and stored in a dry atmosphere, preferably at 4° C. and protected from light.
the sample to be tested is then preferably diluted; this dilution can be between 125 and 1000 times, in particular 150 to 1000 times;
similarly, preferably, test sera or plasmas, called internal standards, which are biological fluids from healthy subjects, preferably sera or plasma from healthy subjects (representative population matched with the patient population at least for sex and age), are used and preferably diluted; this dilution can be between 125 and 1000 times, in particular 150 to 1000 times;
the sample, a reagent blank and preferably also the internal standards, are distributed, preferably in duplicate, in wells of at least one sensitized microtiter plate,
preferably, the plate(s) are incubated; according to a particularly suitable embodiment, they are incubated between 1 h 30 and 2 h 00, preferably between 1 h 45 and 2 h 00, at a temperature of 35 to 39° C.,
preferably, the plate(s) are washed, that is to say, rinsed, preferably several times, with the aim of eliminating all the non-specific proteins and immunoglobulins;
then, anti-human or animal immunoglobulin antibodies, called secondary antibodies, are added that are directed against isotypes A, M or G for humans, A, M, G or E in animals; these secondary antibodies are antibodies directed against the immunoglobulins bound to the antigens present in the wells of the microtiter plates; the secondary antibodies are preferably coupled to an enzyme, and preferably to alkaline peroxidase or phosphatase, to allow a colorimetric reaction to be carried out,
preferably, the plates are incubated again for 1 hour to 2 hours at a temperature between 35 and 39° C.,
preferably, the plates are then washed again, that is to say, several rinses are carried out so as to eliminate what has not been specifically recognized,
preferably, a substrate of the enzyme (H₂O₂ for peroxidase or for alkaline phosphatase) is then applied with a chromogen; the objective of this step is to visualize the immunological reactions. The chromogen can for example be tetramethylbenzidine (TMB) or Diaminobenzidine (DAB); a colorimetric reaction appears; this is all the more intense when there are human or animal immunoglobulins fixed on the antigens present in the wells;
optionally, a new incubate is done for between 10 and 30 minutes at a temperature between 18 and 20° C., preferably protected from light; the reaction is then stopped very preferably in an acid solution;
the optical density of each well of the microtiter plates is then read, preferably using a spectrophotometer; the reading is preferably carried out at 450 nm with a correction alpha at 620 nm or at 650 nm; these optical densities are then preferably recorded in software.

According to a specific algorithm, the OD values of the patient's serum (human or animal) of all the specific markers defining his immunological profile are held up against the distribution of a population of patients suffering from a chronic degenerative pathology versus the distribution of a population of healthy controls. These population distributions depend on a Mann and Whitney U test with a probability associated with a Mann and Whitney U test (P value) threshold of 0.01, on a distribution by percentiles. It is thus possible to detect whether the human being or the animal to which the tested serum and/or plasma belongs is affected by a degenerative disease.

In addition, the method according to the invention also makes it possible to monitor the progression of the disease. The variation of the OD value for the sought antibody or antibodies makes it possible to verify the variation and the progression of the disease, defined by the P value of the Mann and Whitney U test:
If the P value is less than 0.01, defined by the Mann and Whitney U test, of the distribution of the patient population (human or animal) compared to the population distribution of healthy controls, then the level of circulating serum antibodies directed against a bacterial antigen and/or a neoantigen is significantly different from the level of circulating serum antibodies in a population of healthy controls.
If the P value is greater than 0.01, defined by the Mann and Whitney U test, of the distribution of the patient population (human or animal) compared to the population distribution of healthy controls, then the level of circulating serum antibodies directed against a bacterial antigen and/or a neoantigen is identical to the level of circulating serum antibodies in a population of healthy controls. The OD(s) obtained from the patient or animal are distributed by percentile according to the distribution chosen by the designed algorithm. In the case of degenerative pathologies, if the ODs are distributed over the distribution by percentiles of the patient population, then for the antibodies sought according to the invention, this means that it is in fact a degenerative pathology.

Assaying one or more specific antibodies also makes it possible to propose a treatment adapted as a function of the tested antibody or antibodies present in the sample.

According to one of the particular embodiments, the method according to the invention comprises at least the implementation of the following steps:
manufacturing at least one microtiter plate sensitized with the antigen(s) against which the antibodies, the presence of which is to be detected in the sample, are directed, or using at least one microtiter plate already sensitized with these antigens,
distributing the same volume of internal standards, sample and reagent blank on the plate,
adding the secondary antibody or antibodies coupled to an enzyme,
adding an enzyme substrate and a chromogen, waiting for the coloring of the wells,
stopping the coloring reaction,
reading the optical density of the wells using a spectrophotometer at an appropriate wavelength.

In particular, a particularly suitable embodiment of the invention is a method that comprises at least implementing the following steps:
manufacturing at least one microtiter plate sensitized with the antigen(s) against which the antibodies, the presence of which is to be detected in the sample, are directed, or using at least one microtiter plate already sensitized with these antibodies,
diluting the sample to be tested and internal standards,
distributing, in duplicate on the plate, the same quantity of diluted internal standards, diluted sample and reagent blank,
incubating,
washing,
adding the secondary antibody or antibodies coupled to peroxidase or alkaline phosphatase,
incubating,
washing,
adding a substrate and a chromogen,
stopping the reaction in acid solution
reading at 450 nm with a correction alpha at 650 nm or at 620 nm.

The antibodies sought in the context of the method according to the invention, independent of the antigens against which they are directed, can be IgM (immunoglobulins of isotype M), IgA (immunoglobulins of isotype A), or IgG (immunoglobulins of isotype G), and in animals only, IgE (immunoglobulins of isotype E):
IgA antibodies: are the result of mucosal immune activation (intestinal, ENT, skin, bladder),
IgM antibodies: are the result of activation of the current immune system,
IgG antibodies: are the result of activation of the old immune system (memory immunity),
IgE antibodies (in animals only) are the result of the stimulation of mast cells, which release histamine, that is to say, they are the result of contact with allergenic antigens.

The method according to the invention is therefore carried out by immunoassay for the presence of antibodies in the sample, including at least:
one or more antibodies directed against at least one enterobacterium, that is to say, against at least one component of at least one enterobacterium, and
one or more antibodies directed against a tryptophan oxidation product.

In fact, according to the invention, if at least these antibodies are present in the tested biological fluid sample, in particular in the tested serum and/or plasma, that is to say, if the test is positive for at least these antibodies, then this means that the person has a chronic degenerative disease. Enterobacteriaceae and tryptophan oxidation products are involved in the physiological mechanisms at the origin of all chronic degenerative diseases.

Preferably, the method according to the invention comprises the immunoassay for the presence of at least one antibody directed against an enterobacterium (against a bacterial component of an enterobacterium) chosen from the following antibodies:
antibody or antibodies directed against the bacterium Pseudomonas aeruginosa,
antibody or antibodies directed against the bacterium Klebsiella pneumoniae,
antibody or antibodies directed against the bacterium Providencia Rettgeri,
antibody or antibodies directed against the bacterium Citrobacter koserii,
antibody or antibodies directed against the bacterium Enterobacter agglomerans,
antibody or antibodies directed against the bacterium Serratia marcensens,
antibody or antibodies directed against the bacterium Citrobacter freundii,
antibody or antibodies directed against the bacterium Proteus mirabilis
antibody or antibodies directed against the bacterium Escherichia coli,
antibody or antibodies directed against the bacterium Pseudomonas putida.

Gram-negative Enterobacteriaceae have a wall whose three-layer structure is specific to them. This wall is made up, from the outside to the inside, of: an outer membrane, a thin layer of peptidoglycan, and a periplasmic space that surrounds the cytoplasmic membrane. The outer membrane consists of a lipid double layer in which lipopolysaccharide molecules are included. The peptidoglycan forms a stiff, thinner and looser layer than in Gram-positive bacteria. It is composed of linear chains and polysaccharides linked together by peptides. The periplasmic space surrounds the cytoplasmic membrane.

These bacteria are non-pathogenic and can be present on the mucous membranes. However, if they pass through the mucous membranes, they can be the source of chronic pathologies, in particular chronic degenerative pathologies. Their transmucosal passage generates:
activation of the innate immune system,
activation of the adaptive immune system,
non-specific activation of self-reactive clones (superantigens),
direct toxicity (lipopolysaccharides, Pili type IV, exolysin, endotoxins),
activation of enzymatic systems, for example inducible NO synthase, and Indolamine 2,3-dioxygenase (IDO-1).

According to the invention, the presence in the tested biological fluid, in particular in the serum and/or in the plasma, of one or more antibodies directed against these bacteria indicates mucosal hyperpermeability, which participates in the development of degenerative diseases.

Preferably, the method according to the invention comprises immunoassay for the presence in the sample of:
IgG, IGM and/or IgA (and/or IgE for animals) directed against the bacterium Pseudomonas aeruginosa, and/or
IgG, IGM and/or IgA (and/or IgE for animals) directed against the bacterium Pseudomonas aeruginosa, and/or
IgG, IgM and/or IgA (and/or IgE for animals) directed against the bacterium Providencia Rettgeri, and/or
IgG, IgM and/or IgA (and/or IgE for animals) directed against the bacterium Citrobacter koserii, and/or
IgG, IgM and/or IgA (and/or IgE for animals) directed against the bacterium Enterobacter agglomerans, and/or
IgG, IgM and/or IgA (and/or IgE for animals) directed against the bacterium Serratia marcensens, and/or
IgG, IgM and/or IgA (and/or IgE for animals) directed against the bacterium Citrobacter freundii, and/or
IgG, IgM and/or IgA (and/or IgE for animals) directed against the bacterium Proteus mirabilis, and/or
IgG, IgM and/or IgA (and/or IgE for animals) directed against the bacterium Escherichia coli, and/or
IgG, IgM and/or IgA (and/or IgE for animals) directed against the bacterium Pseudomonas putida.

Preferably, the method according to the invention comprises immunoassay for the presence of at least one antibody directed against a tryptophan oxidation product chosen from the following antibodies:
antibody or antibodies directed against 3-hydroxykynurenine,
antibody or antibodies directed against picolinic acid,
antibody or antibodies directed against quinolinic acid,
antibody or antibodies directed against xanthurenic acid,
antibody or antibodies directed against anthranilic acid,
antibody or antibodies directed against 3-hydroxyanthranilic acid,
antibody or antibodies directed against quinaldic acid,
antibody or antibodies directed against kynurenic acid
antibody or antibodies directed against 5-hydroxytryptophan acid,
antibodies directed against 5-HIAA acid (hydroxy indole acetic acid),
antibody or antibodies directed against serotonin,
antibody or antibodies directed against melatonin,
antibody or antibodies directed against 5methoxytryptophol,
antibody or antibodies directed against 5-hydroxytroptophol.

L-tryptophan is metabolized into a large number of by-products: neurotransmitters, neurohormonal active in the nervous system. The best known are serotonin and melatonin by way of tryptophan hydroxylase.

The IDO-1 pathway (Indolamine-2,3-dioxygenase) also metabolizes tryptophan. It is an inducible enzymatic pathway. It is present in microglial neuronal cells and cells of the monocyte-macrophage lineage. There are many molecules that activate the IDO pathway. The main ones are the tumor necrosis factor (TNF)-α, interleukins (IL)-12 and (IL)-18, interferon-γ, microorganisms and their derivatives.

IDO-1 degrades L-tryptophan to N-formylkynurenine. A complex enzyme system then converts N-formylkynurenine to L-kynurenine. The latter is metabolized into different compounds by many enzymes. Kynureninase converts L-Kynurenine to anthranilic acid, which gives 5-hydroxy-anthranilic acid. This acid can then be converted into 3-hydroxyanthranilic acid. L-kynurenine is also degraded into 3-hydroxykynurenine by kynurenine 3-hydroxylase. The hydroxylated form is converted to 3-hydroxyanthranilic acid by a kynureninase. The latter compound is metabolized by 3-hydroxyanthranilic acid oxygenase to an intermediate product that originates in three distinct pathways. It gives either a picolinic acid by the picolinic carboxylase, or a quinolinic acid. L-kynurenine is converted to kynurenine by decarboxylases, or to kynurenic acid by kynurenine aminotransferase. This kynurenic acid is at the origin of the synthesis of quinaldic acid.

The IDO-1 pathway limits the pool of extracellular L-tryptophan, which promotes the replication of a large number of cells, and the tryptophan oxidation products, that is to say, the products resulting from the activation of the IDO-1 pathway, induce apoptotic mechanisms at high concentrations. Their hyperproduction therefore causes apoptosis and cell death.

Preferably, the method according to the invention comprises immunoassay for the presence in the sample of:
IgA (and/or IgE for animals) directed against quinaldic acid/IgA (and/or IgE for animals) directed against kynurenic acid
IgG, IgM and/or IgA (and/or IgE for animals) directed against 3-hydroxykynurenine, and/or
IgG, IgM and/or IgA (and/or IgE for animals) directed against picolinic acid, and/or
IgG, IgM and/or IgA (and/or IgE for animals) directed against quinolinic acid, and/or
IgG, IgM and/or IgA (and/or IgE for animals) directed against xanthurenic acid, and/or
IgG, IgM and/or IgA (and/or IgE for animals) directed against anthranilic acid, and/or
IgG, IgM and/or IgA (and/or IgE for animals) directed against 3-hydroxyanthranilic acid, and/or
IgG, IgM and/or IgA (and/or IgE for animals) directed against quinaldic acid, and/or
IgG, IgM and/or IgA (and/or IgE for animals) directed against kynurenic acid, and/or
IgG, IgM and/or IgA (and/or IgE for animals) directed against 5-hydroxytryptophan acid, and/or
IgG, IgM and/or IgA (and/or IgE for animals) directed against 5-HIAA acid, and/or
IgG, IgM and/or IgA (and/or IgE for animals) directed against serotonin, and/or
IgG, IgM and/or IgA (and/or IgE for animals) directed against melatonin, and/or
IgG, IgM and/or IgA (and/or IgE for animals) directed against 5-methoxytryptophol, and/or
IgG, IgM and/or IgA (and/or IgE for animals) directed against 5-hydroxytroptophol.

Preferably, the method according to the invention comprises immunoassay for the presence in the sample of:
IgG, IgM and/or IgA (and/or IgE for animals) directed against tryptophan.

According to the invention, the presence in the biological fluid of at least one antibody directed against a tryptophan oxidation product, in particular IgA and/or Ig M and/or IgG (and/or IgE for animals), and the presence in the biological fluid of at least one antibody directed against an enterobacterium, in particular IgA and/or Ig M and/or IgG (and/or IgE for animals), necessarily means that the concerned person or animal is affected by a degenerative disease.

In addition (either at the same time or in a separate test) to the immunoassay for the presence in the sample of one or more antibodies directed against at least one enterobacterium and of one or more antibodies directed against a tryptophan oxidation product, the method according to the invention can also comprise the immunoassay for the presence in the sample of one or more other antibodies. This may preferably be:
at least one antibody directed against a lipoperoxidation product, and/or
at least one antibody directed against a nitrated or nitrosylated product, and/or
at least one antibody directed against a catecholamine oxidation product, and/or
at least one antibody directed against a bacterial metabolism product.

Preferably, the method according to the invention comprises the immunoassay for the presence of at least one antibody directed against a fatty acid product potentially at the origin of lipoperoxidation mechanisms or resulting from lipoperoxidation, chosen from the following antibodies:
antibody or antibodies directed against lauric acid,
antibody or antibodies directed against hydroxylauric acid,
antibody or antibodies directed against palmitic acid,
antibody or antibodies directed against myristic acid,
antibody or antibodies directed against oleic acid,
antibody or antibodies directed against a fatty acid having between 6 and 12, preferably between 6 and 10 carbon atoms,
antibody or antibodies directed against a hydroxylated fatty acid having between 6 and 12, preferably between 6 and 10 carbon atoms,
antibody or antibodies directed against malondialdehyde.

NO is the source of many toxic radical species, called reactive oxygen species (ROS). Among these ROSs, mention may in particular be made of lipid peroxides and their decomposition products, aldehydes, but also singlet oxygen and peroxynitrites. ROSs manifest their toxicity when they exceed cellular defense possibilities. These ROSs have an aggressive role. They attack phospholipid membranes, proteins and DNA. When they attack the phospholipid membranes, the radical attack can modify the lipid compounds constituting the cell and thus alter the messages coming from the environment toward the interior of the cell, causing a dysfunction that can be irreversible.

This lipoperoxidation is a cascade reaction that starts with the oxidation of the fatty acid chains constituting phospholipids, preferably unsaturated fatty acids having carbon-carbon double bonds. These unsaturated fatty acids ensure the fluidity of the membranes at best. They become more unstable and more fragile.

Lipoperoxidation is made possible, on the one hand, by the existence of these double bonds of the polyunsaturated fatty acids, which facilitates the delocalization of the free electron, and, on the other hand, by the presence of molecular oxygen, which will easily pair one of its electrons with the delocalized free electron.

There are several types of lipoperoxidation:
a first, very active radical cascade, which will generate the production of free radicals,
the reaction of the O₂₋ superoxide radical with NO, which leads to the formation of very aggressive peroxynitrites that will cause the degradation of fatty acids into hydroperoxides, then into malondialdehyde (small molecule, extremely reactive with amino residues, and normally not present in the body).
another, milder type of lipoperoxidation causes, by hydrolysis, a breaking of the double bonds of the polyunsaturated fatty acids with the formation of diacids, one of the main compounds of which is azelaic acid.

The presence of lipoperoxidation products in humans or animals causes a disruption of the cell membranes. Indeed, they disrupt the action potential of cell membranes, which causes a breakdown of the barrier effect, allowing outside molecules to enter the interior of the cells and causing apoptosis. This in particular means cellular degeneration, and therefore degenerative disease.

Preferably, the method according to the invention comprises immunoassay for the presence in the sample of:
IgA and/or IgM and/or IgG (and/or IgE for animals) directed against lauric acid, and/or
IgA and/or IgM and/or IgG (and/or IgE for animals) directed against hydroxylauric acid, and/or
IgA and/or IgM and/or IgG (and/or IgE for animals) directed against palmitic acid, and/or
IgA and/or IgM and/or IgG (and/or IgE for animals) directed against myristic acid, and/or
IgA and/or IgM and/or IgG (and/or IgE for animals) directed against oleic acid, and/or
IgA and/or IgM and/or IgG (and/or IgE for animals) directed against a fatty acid having between 6 and 12, preferably between 6 and 10 carbon atoms, and/or
IgA and/or IgM and/or IgG (and/or IgE for animals) directed against a hydroxylated fatty acid having between 6 and 12, preferably between 6 and 10 carbon atoms, and/or
IgA and/or IgM and/or IgG (and/or IgE for animals) directed against malondialdehyde.

Preferably, the method according to the invention comprises immunoassay for the presence of at least one antibody directed against a nitrated or nitrosylated product chosen from the following antibodies:
antibody or antibodies directed against NO-Cysteine,
antibody or antibodies directed against NO₂-Tyrosine,
antibody or antibodies directed against NO-Phenylalanine
antibody or antibodies directed against NO-creatine,
antibody or antibodies directed against NO-Tryptophan,
antibody or antibodies directed against NO-methionine,
antibody or antibodies directed against NO-histamine.

Nitric oxide (NO) has a single electron, which gives it very rapid reactivity. It depends on its ability to share its electron with other radicals or metals. In biological media, the synthesis of NO from L-arginine passes through an intermediate, hydroxy-L-arginine (HOA) under the action of NO synthase (NOS). Oxidation at the terminal nitrogen of the guanidine function leads to the formation of NO.

Two types of NOS are present in most vertebrates: so-called constitutive NOS (cNOS) and inducible NOS (iNOS).

Nitrated or nitrosylated products are neoantigens resulting from an overproduction of NO and/or peroxynitrite that results from the activation of inducible NO synthase, mainly in the cells of the innate immune system, by bacteria, viruses, pollutants, etc. The excess of NO and/or peroxynitrite results in their binding to endogenous proteins, which become immunogenic. The endogenous proteins, the structure of which is thus modified, lead to cell apoptosis and consequently degenerative disease.

Preferably, the method according to the invention comprises immunoassay for the presence in the sample of:
IgG and/or IgM and/or IgA (and/or IgE for animals) directed against NO-Cysteine, and/or
IgG and/or IgM and/or IgA and/or IgM (and/or IgE for animals) directed against NO₂-Tyrosine, and/or
IgG and/or IgM and/or IgA (and/or IgE for animals) directed against NO-creatine, and/or
IgG and/or IgM and/or IgA (and/or IgE for animals) directed against NO-Tryptophan, and/or
IgG and/or IgM and/or IgA (and/or IgE for animals) directed against NO-Phenylalanine
IgG and/or IgM and/or IgA (and/or IgE for animals) directed against NO-methionine, and/or
IgG and/or IgM and/or IgA (and/or IgE for animals) directed against NO-histamine.

The method according to the invention can also comprise the immunoassay for the presence of at least one antibody directed against another nitrated or nitrosylated product chosen from the following antibodies:
antibody or antibodies directed against NO-citrulline,
antibody or antibodies directed against NO-asparagine,
antibody or antibodies directed against NO-arginine,
antibody or antibodies directed against NO-phenylalanine.

Preferably, the method according to the invention comprises immunoassay for the presence in the sample of:
IgG and/or IgM and/or IgA (and/or IgE for animals) directed against NO-citrulline, and/or
IgG and/or IgM and/or IgA (and/or IgE for animals) directed against NO-asparagine, and/or
IgG and/or IgM and/or IgA (and/or IgE for animals) directed against NO-arginine, and/or
IgG and/or IgM and/or IgA (and/or IgE for animals) directed against NO-phenylalanine.

Preferably, the method according to the invention comprises immunoassay for the presence of at least one antibody directed against a catecholamine oxidation product chosen from the following antibodies:
antibody or antibodies directed against norepinephrine
antibody or antibodies directed against N-acetylcysteine,
antibody or antibodies directed against homovanillic acid N-acetylcysteine,
antibody or antibodies directed against L-dopa N-acetylcysteine,
antibody or antibodies directed against dopamine N-acetylcysteine,
antibody or antibodies directed against epinephrine N-acetylcysteine.

The catecholamine oxidation products are the result of the production of oxidized and radical compounds (O₂, O₂₋, NO, NO₂).

Their presence in humans or animals causes cell apoptosis by hyperconsumption of energy, and consequently causes degenerative disease.

Preferably, the method according to the invention comprises immunoassay for the presence in the sample of:
IgG and/or IgM and/or IgA (and/or IgE for animals) directed against norepinephrine
IgG and/or IgM and/or IgA (and/or IgE for animals) directed against N-acetylcysteine,
IgG and/or IgM and/or IgA (and/or IgE for animals) directed against homovanillic acid N-acetylcysteine,
IgG and/or IgM and/or IgA (and/or IgE for animals) directed against homovanillic acid N-acetylcysteine,
IgG and/or IgM and/or IgA (and/or IgE for animals) directed against L-dopa N-acetylcysteine,
IgG and/or IgM and/or IgA (and/or IgE for animals) directed against dopamine N-acetylcysteine,
IgG and/or IgM and/or IgA (and/or IgE for animals) directed against epinephrine N-acetylcysteine.

The method according to the invention can also comprise the immunoassay for the presence of at least one antibody directed against a bacterial metabolism product chosen from the following antibodies:
antibody or antibodies directed against succinate,
antibody or antibodies directed against butyrate,
antibody or antibodies directed against acetate,
antibody or antibodies directed against propionate,

These bacterial metabolic compounds reflect bacterial hyperactivity in the mucous membranes.

Preferably, the method according to the invention comprises immunoassay for the presence in the sample of:
IgG and/or IgM and/or IgA (and/or IgE for animals) directed against succinate, and/or
IgG and/or IgM and/or IgA (and/or IgE for animals) directed against butyrate, and/or
IgG and/or IgM and/or IgA (and/or IgE for animals) directed against acetate, and/or
IgG and/or IgM and/or IgA (and/or IgE for animals) directed against propionate.

According to a particular embodiment, the method according to the invention is specifically intended for detecting or monitoring the progression of amyotrophic lateral sclerosis. In this case, the method is preferably carried out by immunoassay for the presence of antibodies in the sample, including at least:
one or more of the following antibodies:
   antibody or antibodies directed against the bacterium Klebsiella pneumoniae,
   antibody or antibodies directed against the bacterium Providencia Rettgeri,
   antibody or antibodies directed against the bacterium Citrobacter koserii,
   antibody or antibodies directed against the bacterium Enterobacter agglomerans,
   antibody or antibodies directed against the bacterium Pseudomonas aeruginosa,
   antibody or antibodies directed against the bacterium Pseudomonas putida,
   antibody or antibodies directed against the bacterium Serratia marcensens,
   antibody or antibodies directed against the bacterium Citrobacter freundii,
and one or more of the following antibodies:
   antibody or antibodies directed against 3-hydroxykynurenine,
   antibody or antibodies directed against picolinic acid,
   antibody or antibodies directed against quinolinic acid,
   antibody or antibodies directed against xanthurenic acid,
   antibody or antibodies directed against anthranilic acid,
   antibody or antibodies directed against 5-hydroxyanthranilic acid,
   antibody or antibodies directed against kynurenic acid,
   antibody or antibodies directed against quinaldic acid,
   antibody or antibodies directed against 5-hydroxytryptophan acid,
   antibody or antibodies directed against 5-HIAA acid,
   antibody or antibodies directed against serotonin,
   antibody or antibodies directed against melatonin,
   antibody or antibodies directed against 5-methoxytryptophol,
   antibody or antibodies directed against 5-hydroxytroptophol,
   antibody or antibodies directed against tryptophan,
and one or more of the following antibodies:
   antibody or antibodies directed against NO-Cysteine,
   antibody or antibodies directed against NO₂-Tyrosine,
   antibody or antibodies directed against NO-creatine,
   antibody or antibodies directed against NO-Tryptophan,
   antibody or antibodies directed against NO-methionine,
   antibody or antibodies directed against NO-histamine,
   antibody or antibodies directed against NO-citrulline,
   antibody or antibodies directed against NO-asparagine,
   antibody or antibodies directed against NO-arginine,
   antibody or antibodies directed against NO-phenylalanine,
   antibody or antibodies directed against lauric acid,
   antibody or antibodies directed against hydroxylauric acid,
   antibody or antibodies directed against palmitic acid,
   antibody or antibodies directed against myristic acid,
   antibody or antibodies directed against oleic acid,
   antibody or antibodies directed against a fatty acid having between 6 and 12, preferably between 6 and 10, carbon atoms,
   antibody or antibodies directed against a hydroxylated fatty acid having between 6 and 12, preferably between 6 and 10, carbon atoms,
   antibody or antibodies directed against homovanillic acid N-acetylcysteine,
   antibody or antibodies directed against L-Dopa N-acetylcysteine,
   antibody or antibodies directed against L-dopamine N-acetylcysteine,
   antibody or antibodies directed against epinephrine N-acetylcysteine,
   antibody or antibodies directed against norepinephrine N-acetylcysteine
   antibody or antibodies directed against succinate,
   antibody or antibodies directed against butyrate,
   antibody or antibodies directed against acetate,
   antibody or antibodies directed against propionate.

According to a particular embodiment, the method according to the invention is specifically intended for detecting or monitoring the progression of Parkinson's disease. In this case, the method is preferably carried out by immunoassay for the presence of antibodies in the sample, including at least:
one or more of the following antibodies:
   antibody or antibodies directed against the bacterium Pseudomonas putida,
   antibody or antibodies directed against the bacterium Hafnia alvei,
   antibody or antibodies directed against the bacterium Pseudomonas aeruginosa,
   antibody or antibodies directed against the bacterium Proteus mirabilis
   antibody or antibodies directed against the bacterium Escherichia coli,
   antibody or antibodies directed against the bacterium Pseudomonas aerofasciens,
and one or more of the following antibodies:
   antibody or antibodies directed against NO-Tryptophan,
   antibody or antibodies directed against NO-methionine,
   antibody or antibodies directed against NO-histamine,
   antibody or antibodies directed against 3-hydroxykynurenine,
   antibody or antibodies directed against picolinic acid,
   antibody or antibodies directed against quinolinic acid,
   antibody or antibodies directed against xanthurenic acid,
   antibody or antibodies directed against anthranilic acid,
   antibody or antibodies directed against 5-hydroxyanthranilic acid,
   antibody or antibodies directed against 5-hydroxytryptophan acid,
   antibody or antibodies directed against 5-HIAA acid,
   antibody or antibodies directed against serotonin,
   antibody or antibodies directed against melatonin,
   antibody or antibodies directed against 5-methoxytryptophol,
   antibody or antibodies directed against 5-hydroxytroptophol,
and one or more of the following antibodies:
   antibody or antibodies directed against NO-Cysteine,
   antibody or antibodies directed against NO₂-Tyrosine,
   antibody or antibodies directed against NO-creatine,
   antibody or antibodies directed against malondialdehyde
   antibody or antibodies directed against norepinephrine N-acetylcysteine,
   antibody or antibodies directed against homovanillic acid N-acetylcysteine,
   antibody or antibodies directed against L-Dopa N-acetylcysteine,
   antibody or antibodies directed against dopamine N-acetylcysteine,
   antibody or antibodies directed against epinephrine N-acetylcysteine.

According to a particular embodiment, the method according to the invention is specifically intended for detecting or monitoring the progression of Alzheimer's disease. In this case, the method is preferably carried out by immunoassay for the presence of antibodies in the sample, including at least:
one or more of the following antibodies:
   antibody or antibodies directed against the bacterium Pseudomonas putida,
   antibody or antibodies directed against the bacterium Hafnia alvei,
   antibody or antibodies directed against the bacterium Pseudomonas aeruginosa,
   antibody or antibodies directed against the bacterium Proteus mirabilis
   antibody or antibodies directed against the bacterium Escherichia coli,
   antibody or antibodies directed against the bacterium Pseudomonas aerofasciens,
and one or more of the following antibodies:
   antibody or antibodies directed against 3-hydroxykynurenine,
   antibody or antibodies directed against picolinic acid,
   antibody or antibodies directed against quinolinic acid,
   antibody or antibodies directed against xanthurenic acid,
   antibody or antibodies directed against anthranilic acid,
   antibody or antibodies directed against 5-hydroxyanthranilic acid,
   antibody or antibodies directed against 5-hydroxytryptophan acid,
   antibody or antibodies directed against 5-HIAA acid,
   antibody or antibodies directed against serotonin,
   antibody or antibodies directed against melatonin,
   antibody or antibodies directed against 5-methoxytryptophol,
   antibody or antibodies directed against 5-hydroxytroptophol,
and one or more of the following antibodies:
   antibody or antibodies directed against NO-Cysteine,
   antibody or antibodies directed against NO₂-Tyrosine,
   antibody or antibodies directed against NO-creatine,
   antibody or antibodies directed against NO-Tryptophan,
   antibody or antibodies directed against NO-methionine,
   antibody or antibodies directed against NO-histamine,
   antibody or antibodies directed against NO-citrulline.

To implement the ex vivo method for detecting and/or monitoring the progression of a disease, the invention also relates to diagnostic kits.

In particular, the object of the invention is a kit for use thereof in the detection or monitoring of the progression of a chronic degenerative disease, in a sample of biological fluid, in particular in a sample of human or animal biological fluid comprising at least the antigen(s) against which the antibodies, the presence of which is to be detected in the sample, are directed, namely preferably one or more antibodies chosen from:
antibody or antibodies directed against NO-Cysteine,
antibody or antibodies directed against NO₂-Tyrosine,
antibody or antibodies directed against NO-creatine,
antibody or antibodies directed against NO-Tryptophan,
antibody or antibodies directed against NO-methionine,
antibody or antibodies directed against NO-histamine,
antibody or antibodies directed against NO-citrulline,
antibody or antibodies directed against NO-asparagine,
antibody or antibodies directed against NO-arginine,
antibody or antibodies directed against NO-phenylalanine,
antibody or antibodies directed against 3-hydroxykynurenine,
antibody or antibodies directed against picolinic acid,
antibody or antibodies directed against quinolinic acid,
antibody or antibodies directed against xanthurenic acid,
antibody or antibodies directed against anthranilic acid,
antibody or antibodies directed against 3-hydroxyanthranilic acid,
antibody or antibodies directed against 5-HIAA acid,
antibody or antibodies directed against serotonin,
antibody or antibodies directed against melatonin AS,
antibody or antibodies directed against melatonin AG,
antibody or antibodies directed against 5-methoxytryptophol,
antibody or antibodies directed against 5-hydroxytroptophol,
antibody or antibodies directed against tryptophan,
antibody or antibodies directed against kynurenic acid,
antibody or antibodies directed against quinaldic acid,
antibody or antibodies directed against lauric acid,
antibody or antibodies directed against hydroxylauric acid,
antibody or antibodies directed against palmitic acid,
antibody or antibodies directed against myristic acid,
antibody or antibodies directed against oleic acid,
antibody or antibodies directed against a fatty acid having between 6 and 12, preferably between 6 and 10 carbon atoms,
antibody or antibodies directed against a hydroxylated fatty acid having between 6 and 12, preferably between 6 and 10 carbon atoms,
antibody or antibodies directed against creatine,
antibody or antibodies directed against alanine,
antibody or antibodies directed against homovanillic acid N-acetylcysteine,
antibody or antibodies directed against L-dopa N-acetylcysteine,
antibody or antibodies directed against L-dopamine N-acetylcysteine,
antibody or antibodies directed against norepinephrine N-acetylcysteine,
antibody or antibodies directed against epinephrine N-acetylcysteine
antibody or antibodies directed against the bacterium Klebsiella pneumoniae,
antibody or antibodies directed against the bacterium Providencia Rettgeri,
antibody or antibodies directed against the bacterium Citrobacter koserii,
antibody or antibodies directed against the bacterium Enterobacter agglomerans,
antibody or antibodies directed against the bacterium Pseudomonas aeruginosa,
antibody or antibodies directed against the bacterium Pseudomonas putidal,
antibody or antibodies directed against the bacterium Serratia marcensens,
antibody or antibodies directed against the bacterium Citrobacter freundii,
antibody or antibodies directed against succinate,
antibody or antibodies directed against butyrate,
antibody or antibodies directed against acetate,
antibody or antibodies directed against propionate,
antibody or antibodies directed against malondialdehyde—antibody or antibodies directed against acetylcholine
antibody or antibodies directed against norepinephrine N-acetylcysteine,
antibody or antibodies directed against homovanillic acid N-acetylcysteine,
antibody or antibodies directed against L-dopa N-acetylcysteine,
antibody or antibodies directed against dopamine N-acetylcysteine,
antibody or antibodies directed against epinephrine N-acetylcysteine.
antibody or antibodies directed against the bacterium Hafnia alvei,
antibody or antibodies directed against the bacterium Proteus mirabilis
antibody or antibodies directed against the bacterium Escherichia coli,
antibody or antibodies directed against the bacterium Pseudomonas aerofasciens.

Preferably, the kit according to the invention comprises at least:
the antigen(s) and/or neoantigen(s) against which the antibodies, the presence of which is to be detected in the sample, are directed, preferably coupled to a protein when they are not bacteria, and a microtiter plate intended to be sensitized with the antigen(s) and/or neoantigen(s) against which the antibodies, the presence of which is to be detected in the sample, are directed, or a microtiter plate already sensitized with the antigen(s) and/or neoantigen(s) against which the antibodies, the presence of which is to be detected in the sample, are directed, or a strip or stick already sensitized with the antigen(s) and/or neoantigen(s) against which the antibodies, the presence of which is to be detected in the sample, are directed, and
the standards making it possible to assess the quality of the test and to define the distribution of the population of patients with a pathology by percentiles, and/or
buffers and solutions suitable for performing an ELISA test, and/or
the secondary antibody or antibodies in solution, said secondary antibody or antibodies corresponding to the isotypies of the primary antibodies, the presence of which is to be detected in the sample and with the same isotypy, and/or
dilution and washing buffers, and/or
development buffers, and/or
a stop solution.

The invention is now illustrated by examples and results of implementing methods according to the invention.

For each example, the protocol was as follows:
collection of the patient's plasma
implementation of the method according to the invention
summary of the obtained results with, in the graphs, the distribution of the patient population compared to the distribution of the control population. If the P value is <0.01, then the distribution of the patient population is significantly different from the distribution of the control population. This means that the patient population has a higher concentration of circulating serum antibodies directed against a given marker.

Each example makes it possible to define, by percentiles, the distribution of a population of patients suffering from a chronic degenerative pathology. The level of circulating antibodies directed against an antigen and/or a neoantigen is significantly different from that of the population of healthy controls if the P value is less than 0.01.

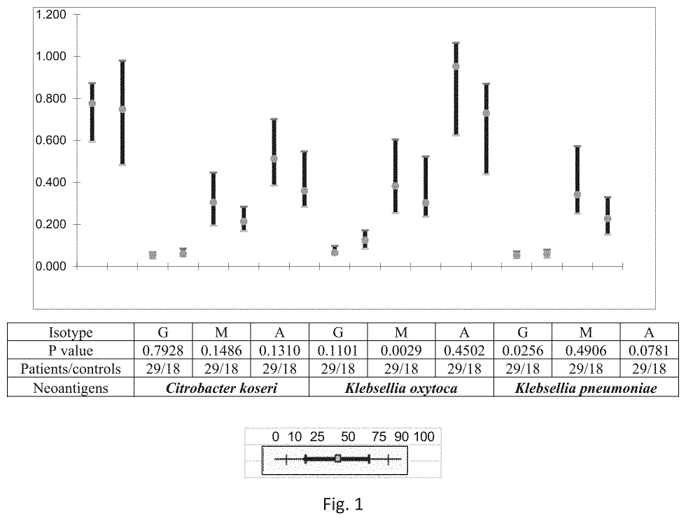

The results of the detection of circulating antibodies directed against Citrobacter koseri in IgA, IgM and IgG, Klebsellia oxytoca in IgA, IgM and IgG and Klebsellia pneumoniae in IgA, IgM and IgG, during the progression of Amyotrophic Lateral Sclerosis are shown in FIG. 1, as a distribution of a population of ALS patients; the midpoint represents 50% of the population.

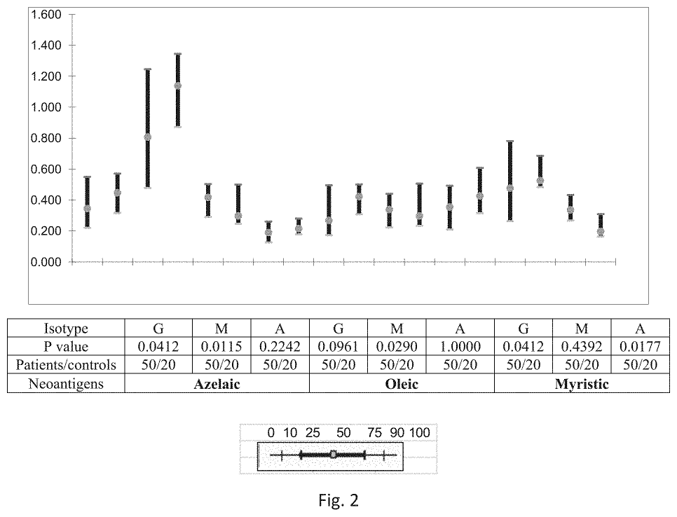

The results of the detection of circulating antibodies directed against azelaic acid in IgA, IgM and IgG, oleic acid in IgA, IgM and IgG and myristic acid in IgA, IgM and IgG, during the progression of Amyotrophic Lateral Sclerosis are shown in FIG. 2, as a distribution of a population of ALS patients; the midpoint represents 50% of the population.

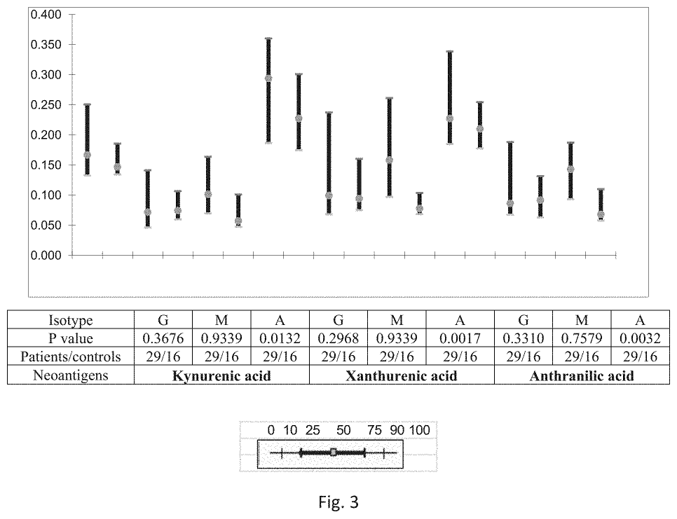

The results of the detection of circulating antibodies directed against kynurenic acid in IgA, IgM and IgG, xanthurenic acid in IgA, IgM and IgG and anthranilic acid in IgA, IgM and IgG, during the progression of Amyotrophic Lateral Sclerosis are shown in FIG. 3, as a distribution of a population of ALS patients; the midpoint represents 50% of the population.

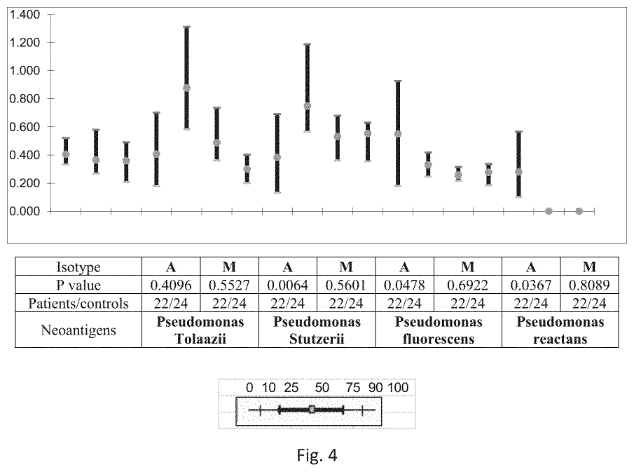

The results of the detection of circulating antibodies directed against Pseudomonas Tolaazii in IgA and IgM, Pseudomonas Stutzerii in IgA and IgM, Pseudomonas fluorescens in IgA and IgM and Pseudomonas reactans in IgA and IgM, during the progression of Parkinson's disease are shown in FIG. 4, as a distribution of a population of Parkinson's disease patients; the midpoint represents 50% of the population.

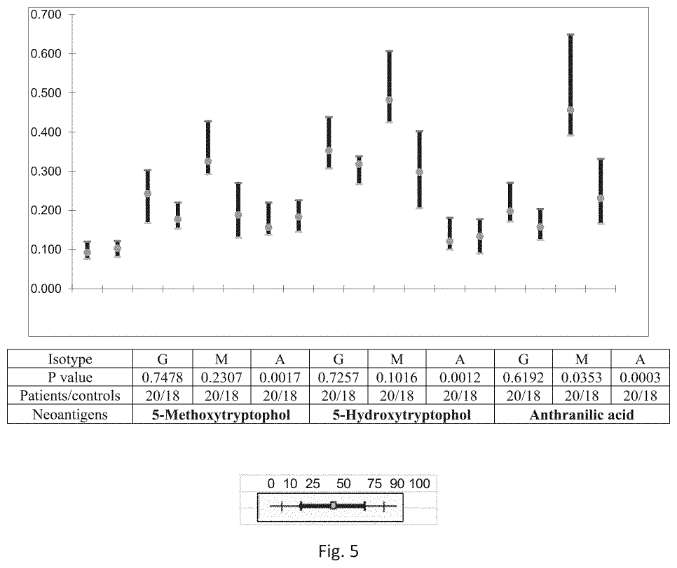

The results of the detection of circulating antibodies directed against 5-methoxytryptophol in IgA, IgG and IgM, 5-hydroxytryptophol in IgA, IgG and IgM, and anthranilic acid in IgA, IgG and IgM, during the progression of Parkinson's disease are shown in FIG. 5, as a distribution of a population of Parkinson's disease patients; the midpoint represents 50% of the population.

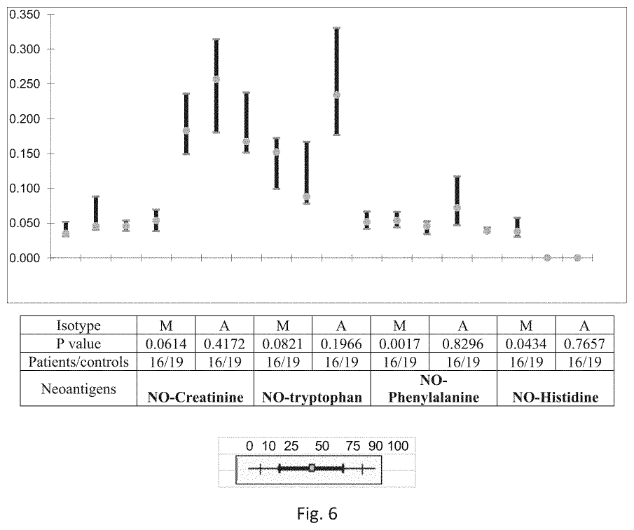

The results of the detection of circulating antibodies directed against NO-creatinine in IgA and IgM, NO-tryptophan in IgA and IgM, NO-phenylalanine in IgA and IgM and NO-histidine in IgA and IgM, during the progression of Parkinson's disease are shown in FIG. 6, as a distribution of a population of Parkinson's disease patients; the midpoint represents 50% of the population.

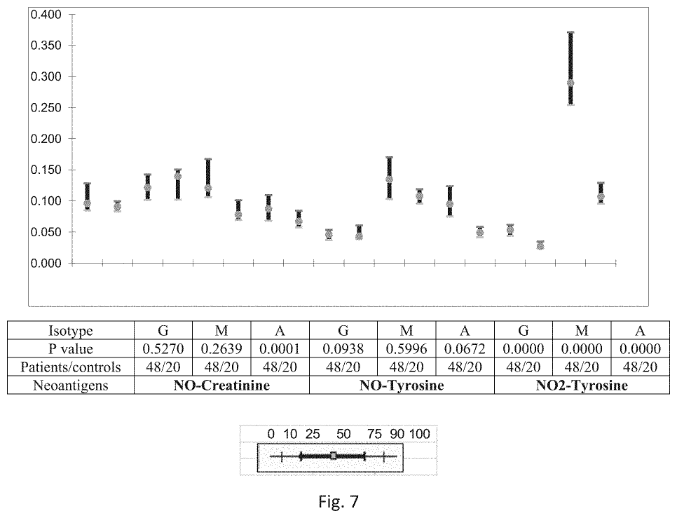

The results of the detection of circulating antibodies directed against NO-creatinine in IgA, IgG and IgM, NO-tyrosine in IgA, IgG and IgM, and NO2 tyrosine in IgA, IgG and IgM, during the progression of Alzheimer's disease are shown in FIG. 7, as a distribution of a population of Alzheimer's disease patients; the midpoint represents 50% of the population.

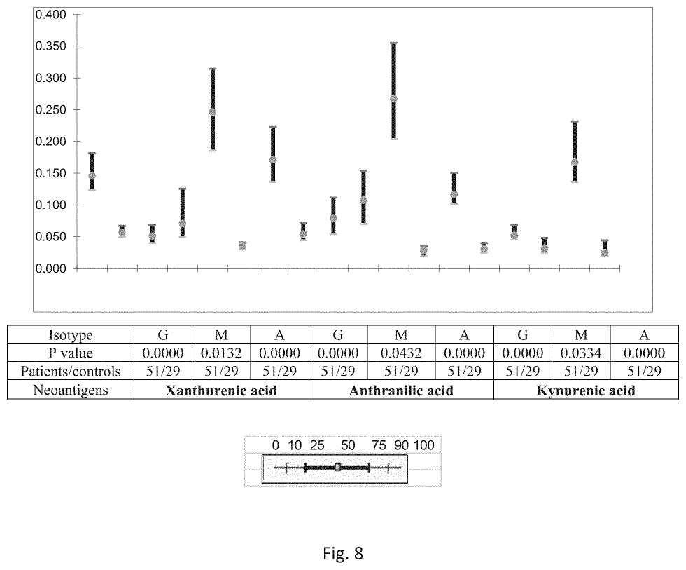

The results of the detection of circulating antibodies directed against xanthurenic acid in IgA, IgG and IgM, anthranilic acid in IgA, IgG and IgM, and kynurenic acid in IgA, IgG and IgM, during the progression of Alzheimer's disease are shown in FIG. 8, as a distribution of a population of Alzheimer's disease patients; the midpoint represents 50% of the population.

1. An ex vivo method for detecting or monitoring the progression of a chronic degenerative disease, in a sample of human or animal biological fluid, by immunoassay for the presence of antibodies in the sample, including at least:
one or more antibodies directed against at least one enterobacterium, chosen from the following antibodies:
antibody or antibodies directed against the bacterium Pseudomonas putida,
antibody or antibodies directed against the bacterium Hafnia alvei,
antibody or antibodies directed against the bacterium Pseudomonas aeruginosa,
antibody or antibodies directed against the bacterium Citrobacter koseri
antibody or antibodies directed against the bacterium Klebsiella pneumoniae,
antibody or antibodies directed against the bacterium Providencia Rettgeri,
antibody or antibodies directed against the bacterium Citrobacter koserii,
antibody or antibodies directed against the bacterium Enterobacter agglomerans,
antibody or antibodies directed against the bacterium Proteus mirabilis
antibody or antibodies directed against the bacterium Escherichia coli,
antibody or antibodies directed against the bacterium Pseudomonas aerofasciens,
antibody or antibodies directed against the bacterium Serratia marcensens,
antibody or antibodies directed against the bacterium Citrobacter freundii, and
one or more antibodies directed against a tryptophan oxidation product, chosen from the following antibodies:
antibody or antibodies directed against 3-hydroxykynurenine,
antibody or antibodies directed against picolinic acid,
antibody or antibodies directed against quinolinic acid,
antibody or antibodies directed against xanthurenic acid,
antibody or antibodies directed against anthranilic acid,
antibody or antibodies directed against 5-hydroxyanthranilic acid,
antibody or antibodies directed against 5-hydroxytryptophan acid,
antibody or antibodies directed against 5-HIAA acid,
antibody or antibodies directed against serotonin,
antibody or antibodies directed against melatonin,
antibody or antibodies directed against 5-methoxytryptophol,
antibody or antibodies directed against 5-hydroxytroptophol,
antibody or antibodies directed against tryptophan,
antibody or antibodies directed against kynurenic acid,
antibody or antibodies directed against quinaldic acid.

2. The ex vivo method of detecting or monitoring the progression of a chronic degenerative disease according to claim 1, characterized in that the sample is a sample of human or animal serum or plasma.

3. The ex vivo method for detecting or monitoring the progression of a chronic degenerative disease according to claim 1, characterized in that it also comprises the immunoassay in the sample for the presence of:
at least one antibody directed against a product at the origin of or resulting from lipoperoxidation, chosen from the following antibodies:
antibody or antibodies directed against lauric acid,
antibody or antibodies directed against hydroxylauric acid,
antibody or antibodies directed against palmitic acid,
antibody or antibodies directed against myristic acid,
antibody or antibodies directed against oleic acid,
antibody or antibodies directed against a fatty acid having between 6 and 10 carbon atoms,
antibody or antibodies directed against a hydroxylated fatty acid having between 6 and 10 carbon atoms,
antibody or antibodies directed against malondialdehyde, and/or
at least one antibody directed against a nitrated or nitrosylated product, chosen from the following antibodies:
antibody or antibodies directed against NO-Cysteine,
antibody or antibodies directed against NO₂-Tyrosine,
antibody or antibodies directed against NO-creatine,
antibody or antibodies directed against NO-Tryptophan,
antibody or antibodies directed against NO-methionine,
antibody or antibodies directed against NO-histamine,
antibody or antibodies directed against NO-citrulline,
antibody or antibodies directed against NO-asparagine,
antibody or antibodies directed against NO-arginine,
antibody or antibodies directed against NO-phenylalanine, and/or
at least one antibody directed against a catecholamine oxidation product, chosen from the following antibodies:
antibody or antibodies directed against norepinephrine N-acetylcysteine,
antibody or antibodies directed against homovanillic acid N-acetylcysteine,
antibody or antibodies directed against L-Dopa N-acetylcysteine,
antibody or antibodies directed against norepinephrine N-acetylcysteine,
antibody or antibodies directed against epinephrine N-acetylcysteine, and/or
at least one antibody directed against a bacterial metabolism product, chosen from the following antibodies:
antibody or antibodies directed against succinate,
antibody or antibodies directed against butyrate,
antibody or antibodies directed against acetate,
antibody or antibodies directed against propionate.

4. The ex vivo method for detecting or monitoring the progression of a chronic degenerative disease according to claim 1, characterized in that the antibodies are immunoglobulins A and/or immunoglobulins G and/or immunoglobulins M and/or, for animals only, immunoglobulins E.

5. The ex vivo method for detecting or monitoring the progression of a chronic degenerative disease according to claim 1, characterized in that said disease is chosen from amyotrophic lateral sclerosis, Parkinson's disease and Alzheimer's disease.

6. The ex vivo method for detecting or monitoring the progression of a chronic degenerative disease according to claim 1, characterized in that said disease is amyotrophic lateral sclerosis and in that the method is carried out by immunoassay for the presence of antibodies in the sample, including at least:
one or more of the following antibodies:
antibody or antibodies directed against the bacterium Klebsiella pneumoniae,
antibody or antibodies directed against the bacterium Providencia Rettgeri,
antibody or antibodies directed against the bacterium Citrobacter koserii,
antibody or antibodies directed against the bacterium Enterobacter agglomerans,
antibody or antibodies directed against the bacterium Pseudomonas aeruginosa,
antibody or antibodies directed against the bacterium Pseudomonas putida,
antibody or antibodies directed against the bacterium Serratia marcensens,
antibody or antibodies directed against the bacterium Citrobacter freundii, and
one or more of the following antibodies:
antibody or antibodies directed against 3-hydroxykynurenine,
antibody or antibodies directed against picolinic acid,
antibody or antibodies directed against quinolinic acid,
antibody or antibodies directed against xanthurenic acid,
antibody or antibodies directed against anthranilic acid,
antibody or antibodies directed against 5-hydroxyanthranilic acid,
antibody or antibodies directed against kynurenic acid,
antibody or antibodies directed against quinaldic acid,
antibody or antibodies directed against 5-hydroxytryptophan acid,
antibody or antibodies directed against 5-HIAA acid,
antibody or antibodies directed against serotonin,
antibody or antibodies directed against melatonin,
antibody or antibodies directed against 5-methoxytryptophol
antibody or antibodies directed against 5-hydroxytroptophol,
antibody or antibodies directed against tryptophan, and
one or more of the following antibodies:
antibody or antibodies directed against NO-Cysteine,
antibody or antibodies directed against NO₂-Tyrosine,
antibody or antibodies directed against NO-creatine,
antibody or antibodies directed against NO-Tryptophan,
antibody or antibodies directed against NO-methionine,
antibody or antibodies directed against NO-histamine,
antibody or antibodies directed against NO-citrulline,
antibody or antibodies directed against NO-asparagine,
antibody or antibodies directed against NO-arginine,
antibody or antibodies directed against NO-phenylalanine,
antibody or antibodies directed against lauric acid,
antibody or antibodies directed against hydroxylauric acid,
antibody or antibodies directed against palmitic acid,
antibody or antibodies directed against myristic acid,
antibody or antibodies directed against oleic acid,
antibody or antibodies directed against a fatty acid having between 6 and 10 carbon atoms,
antibody or antibodies directed against a hydroxylated fatty acid having between 6 and 10 carbon atoms,
antibody or antibodies directed against homovanillic acid N-acetylcysteine,
antibody or antibodies directed against L-Dopa N-acetylcysteine,
antibody or antibodies directed against L-dopamine N-acetylcysteine,
antibody or antibodies directed against epinephrine N-acetylcysteine,
antibody or antibodies directed against norepinephrine N-acetylcysteine
antibody or antibodies directed against succinate,
antibody or antibodies directed against butyrate,
antibody or antibodies directed against acetate,
antibody or antibodies directed against propionate.

7. The ex vivo method for detecting or monitoring the course of a chronic degenerative disease according to claim 1, characterized in that said disease is Parkinson's disease and in that the method is carried out by immunoassay for the presence of antibodies in the sample, including at least:
one or more of the following antibodies:
antibody or antibodies directed against the bacterium Pseudomonas putida,
antibody or antibodies directed against the bacterium Hafnia alvei,
antibody or antibodies directed against the bacterium Pseudomonas aeruginosa,
antibody or antibodies directed against the bacterium Proteus mirabilis
antibody or antibodies directed against the bacterium Escherichia coli,
antibody or antibodies directed against the bacterium Pseudomonas aerofasciens, and
one or more of the following antibodies:
antibody or antibodies directed against NO-Tryptophan,
antibody or antibodies directed against NO-methionine,
antibody or antibodies directed against NO-histamine,
antibody or antibodies directed against 3-hydroxykynurenine,
antibody or antibodies directed against picolinic acid,
antibody or antibodies directed against quinolinic acid,
antibody or antibodies directed against xanthurenic acid,
antibody or antibodies directed against anthranilic acid,
antibody or antibodies directed against 5-hydroxyanthranilic acid,
antibody or antibodies directed against 5-hydroxytryptophan acid,
antibody or antibodies directed against 5-HIAA acid,
antibody or antibodies directed against serotonin,
antibody or antibodies directed against melatonin,
antibody or antibodies directed against 5-methoxytryptophol,
antibody or antibodies directed against 5-hydroxytroptophol,
and one or more of the following antibodies:
antibody or antibodies directed against NO-Cysteine,
antibody or antibodies directed against NO₂-Tyrosine,
antibody or antibodies directed against NO-creatine,
antibody or antibodies directed against malondialdehyde
antibody or antibodies directed against norepinephrine N-acetylcysteine,
antibody or antibodies directed against homovanillic acid N-acetylcysteine,
antibody or antibodies directed against L-Dopa N-acetylcysteine,
antibody or antibodies directed against dopamine N-acetylcysteine,
antibody or antibodies directed against epinephrine N-acetylcysteine.

8. The ex vivo method for detecting or monitoring the course of a chronic degenerative disease according to claim 1, characterized in that said disease is Alzheimer's disease and in that the method is carried out by immunoassay for the presence of antibodies in the sample, including at least:
one or more of the following antibodies:
antibody or antibodies directed against the bacterium Pseudomonas putida,
antibody or antibodies directed against the bacterium Hafnia alvei,
antibody or antibodies directed against the bacterium Pseudomonas aeruginosa,
antibody or antibodies directed against the bacterium Proteus mirabilis
antibody or antibodies directed against the bacterium Escherichia coli,
antibody or antibodies directed against the bacterium Pseudomonas aerofasciens, and
one or more of the following antibodies:
antibody or antibodies directed against 3-hydroxykynurenine,
antibody or antibodies directed against picolinic acid,
antibody or antibodies directed against quinolinic acid,
antibody or antibodies directed against xanthurenic acid,
antibody or antibodies directed against anthranilic acid,
antibody or antibodies directed against 5-hydroxyanthranilic acid,
antibody or antibodies directed against 5-hydroxytryptophan acid,
antibody or antibodies directed against 5-HIAA acid,
antibody or antibodies directed against serotonin,
antibody or antibodies directed against melatonin,
antibody or antibodies directed against 5-methoxytryptophol,
antibody or antibodies directed against 5-hydroxytroptophol, and
one or more of the following antibodies:
antibody or antibodies directed against NO-Cysteine,
antibody or antibodies directed against NO₂-Tyrosine,
antibody or antibodies directed against NO-creatine,
antibody or antibodies directed against NO-Tryptophan,
antibody or antibodies directed against NO-methionine,
antibody or antibodies directed against NO-histamine,
antibody or antibodies directed against NO-citrulline.

9. The ex vivo method for detecting or monitoring the progression of a chronic disease according to claim 1, characterized in that the immunoassay is carried out by implementing an immuno-enzymatic method.

10. The ex vivo method of detecting or monitoring the progression of a chronic disease according to claim 1, characterized in that it comprises at least the implementation of the following steps:
manufacturing at least one microtiter plate sensitized with the antigen(s) against which the antibodies, the presence of which is to be detected in the sample, are directed, or using at least one microtiter plate already sensitized with these antigens,
distributing the same quantity of internal standards, sample and reagent blank on the plate,
adding the secondary antibody or antibodies coupled to an enzyme,
adding an enzyme substrate and a chromogen, waiting for the coloring of the wells,
stopping the coloring reaction, and
reading the optical density of the wells using a spectrophotometer at an appropriate wavelength.

11. The ex vivo method of detecting or monitoring the progression of a chronic disease according to the preceding claim, characterized in that it comprises at least the implementation of the following steps:
manufacturing at least one microtiter plate sensitized with the antigen(s) against which the antibodies, the presence of which is to be detected in the sample, are directed, or using at least one microtiter plate already sensitized with these antigens,
diluting the sample to be tested and internal standards,
distributing, in duplicate on the plate, the same volume of diluted internal standards, diluted sample and reagent blank,
incubating,
washing,
adding the secondary antibody or antibodies coupled to alkaline peroxidase or phosphatase or
incubating,
washing,
adding a substrate and a chromogen,
stopping the reaction in acidic solution, and
reading at 450 nm with a correction alpha at 620 nm.

12. A kit for use in a method for detecting or monitoring the progression of a chronic degenerative disease according to one of the preceding claims, characterized in that it comprises at least the neoantigen(s) against which the antibodies, the presence of which is to be detected in the sample, are directed.

13. The kit according to the preceding claim, characterized in that it comprises at least:
a microtiter plate sensitized with the antigen(s) against which the antibodies, the presence of which is to be detected in the sample, is directed,
buffers and solutions, and
the secondary antibody or antibodies in solution, secondary antibody or antibodies corresponding to the antibodies whereof the presence is to be detected in the sample.
